(19) United States
(12) Patent Application Publication
(10) Pub. No.: US 2012/0220912 A1
(43) Pub. Date: Aug. 30, 2012

(54) FORESKIN PATCH AND FIXATOR FOR THE CIRCUMCISION SURGERY

(76) Inventor: Jianzhong Shang, Wuhu City (CN)

(21) Appl. No.: 13/504,927

(22) PCT Filed: Oct. 25, 2010
(86) PCT No.: PCT/CN2010/001675

§ 371 Date: Apr. 27, 2012

(30) Foreign Application Priority Data
Nov. 3, 2009 (CN) 200920266030.1

(51) Int. Cl. A61B 17/08; A61F 13/02

(52) U.S. Cl. 602/44; 602/54; 602/47; 606/215

(57) ABSTRACT

A foreskin patch and a fixator for use in circumcision surgery. The foreskin patch (1) is in the form of an elongated strip, having an adhesive surface (11) on one side, and a ventilation structure (12). The ventilation structure comprises a plurality of breather holes or a breathable mesh. The fixator (2) comprises an annular sleeve, which has an accommodation space (21) to accommodate a penis. A plurality of fixing pins (22) are provided on the outer surface of the fixator. The fixing pins are needle-like structures vertically located on the outer surface of the fixator. The fixator is integrally formed from materials absorbable by human body.

### CLAIM OF PRIORITY

This application is a national stage application under 35 U.S.C. 371, claiming priority to International patent application number PCT/CN2010/001675, having an international filing date of Oct. 25, 2010, which claims priority to Chinese patent application number 200920266030.1, filed on Nov. 3, 2009, owned by the inventor of the present invention.

### BACKGROUND

I. Field of the Invention

The invention relates to a type of medical apparatus, especially the provision of a type of foreskin patch and a fixator for use in circumcision surgery.

II. Background and Prior Art

Redundant prepuce or phimosis is one of the origins of male urinary system infection and sexually transmitted diseases. Redundant prepuce or phimosis can cause urinary tract infection that will lead to chronic prostatitis, which will incur a series of symptoms like back pain, impotence, prospermia, etc. Therefore, removing redundant foreskin or phimosis is known as a good measure to prevent these sort of diseases.

Regarding the removal of redundant foreskin or phimosis, otherwise known as circumcision, there have been numerous medical advances over the years, but a circumcision operation will inevitably lead to bleeding, pain and hospitalization. Circumcision is one of the traditional measures to treat phimosis and redundant prepuce, performed by medical specialists such as urologists. Typically, circumcision involves three surgical steps comprising redundant foreskin removal, hemostasia, and applying an apposition suture at the incisal edge. After the operation, the patient generally cannot walk, and he will typically feel intolerable pain at each dressing change and further may suffer great pain when stitches are removed. Furthermore, if hemostasis by ligation is done by halves, it may cause preputial hematoma, requiring perhaps another surgical treatment. Although laser and high-frequency electric-knife technology has been used in foreskin circumcisions, their main advantage is only that they eliminate the use of scissors to make incisions and cauterize the point of bleeding. Such a therapeutic method is likely to subject the patient to tissular burn and infection, while the cost for infection prevention is very high. Judging from comparisons among various provinces and cities, whenever laser circumcision is performed, antibiotic treatment after the operation is common for 5-7 days, plus additional antibiotics are usually taken orally for an addition 5-7 days, while the cost generally ranges from 150 to 360 yuan, with the total cost of treatment is at least 1000-1500 yuan.

In addition to the above-mentioned method of laser circumcision, using an electric-knife for circumcising a penis is very common now. Similar to the laser circumcision, it can remove redundant foreskin from the glans to achieve an appropriate length of foreskin. However, the capillary vessel at the wound site typically heals slowly, causing discomfort at the site of the foreskin cut. The risk of infection in such a circumstance is high, with an increased likelihood of pain for the patient.

FIG. 1 illustrates one, prior-art method of performing circumcision surgery. The steps involved in this kind of surgery comprise: removing a middle section of redundant foreskin, bringing the two foreskin edges together, and then suturing them together to achieve a desired foreskin length. In this way, both sides of the capillary vessel at the wound are in contact and, thus, heal quickly.

However, this form of surgery requires a doctor to stitch the joint 30 of the two sides of the foreskin. The surgery is very difficult and it is hard to achieve a good result with respect to healing of the foreskin.

### SUMMARY OF THE INVENTION

The main purpose of this invention lies in overcoming the deficiencies of the existing technology and providing a foreskin patch and a fixator for circumcision surgery.

In order to realize the abovementioned purpose, this invention adopts a technical solution in the form of an apparatus comprising a foreskin patch and a fixator, wherein the foreskin patch is in the form of an elongated strip, having an adhesive surface on one side, and has a ventilation structure. The ventilation structure comprises a plurality of breather holes or breathable meshes. The fixator comprises an annular sleeve, which has an accommodation space to accommodate penis. A plurality of fixing pins are provided on the outer surface of the fixator. The fixing pins are needle-like structures vertically located on the outer surface of the fixator. The fixator is integrally formed from materials absorbable by human body.

The advantageous effects of the invention are as follows:

1. The foreskin patch is used in conjunction with the fixator. As a result, there is no need to use stitches during the circumcision surgery. Therefore, the surgical process is simplified, and the incision will heal to have the best visual effects.

2. The ventilation structure on the foreskin patch, e.g., the breather holes or breathable meshes, can reduce the high risk of infection.

3. The fixing pins on the outer surface of the fixator can secure the foreskin of both sides of the incision to bring them close together with each other and the plaster patch can easily be placed on the foreskin. The structure of the fixator is simple and reliable.

4. The fixator is integrally formed from materials absorbable by human body. There is no need to remove the fixator after surgery.

Further novel features and other objects of the present invention will become apparent from the following detailed description, discussion and claims, taken in conjunction with the drawings.

### BRIEF DESCRIPTION OF THE DRAWINGS

FIG. 1 illustrates a penis circumcised using conventional circumcision method;

FIG. 2A illustrates one embodiment of a foreskin patch of the present invention;

FIG. 2B illustrates another embodiment of the foreskin patch of FIG. 1;

FIG. 2C illustrates a side view of the foreskin patch of the present invention;

FIG. 3A illustrates a fixator used in conjunction with the foreskin patch shown in FIGS. 2A-2C;

FIG. 3B illustrates the side view of the fixator shown in FIG. 3A;

FIG. 3C illustrates an enlarged view of the surface of the fixator shown in FIG. 3A;

FIG. 4 illustrates a perspective view of the fixator used to secure two circumferential foreskin edges together after removal of a middle section of foreskin; and

FIG. 5 illustrates a perspective view of the foreskin patch placed over the fixator shown in FIG. 4 after removal of the middle section of foreskin.

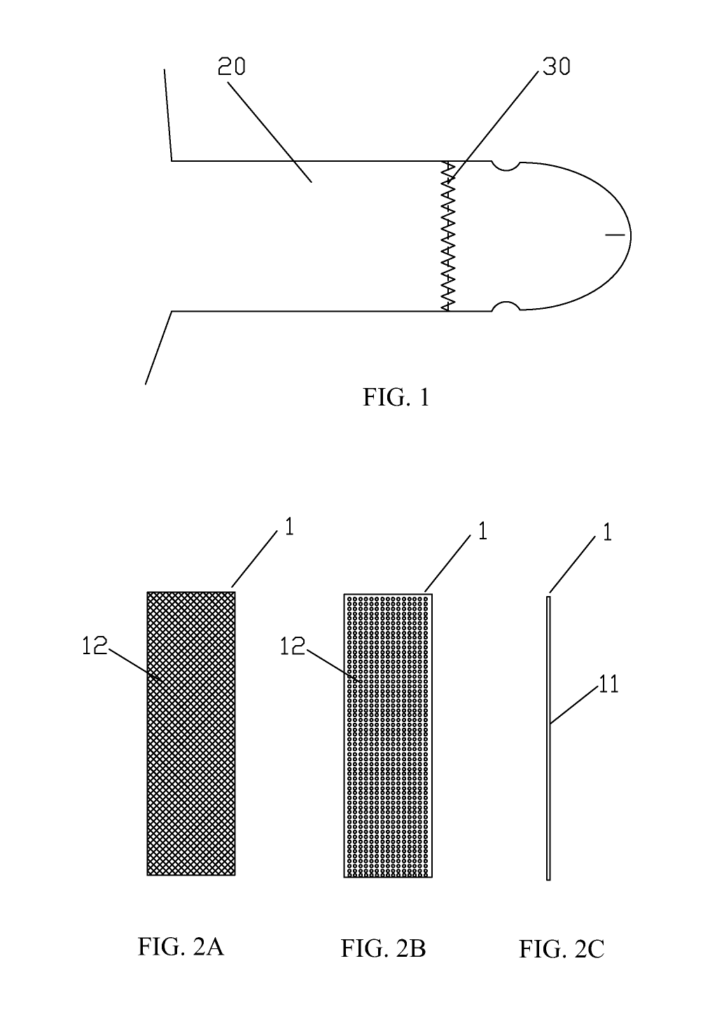

### DETAILED DESCRIPTION

The foreskin patch and the fixator mentioned in this invention used in conjunction with each other. In the surgical field of circumcision, there is prior art that teaches or suggests the use of these two devices together to heal the foreskin that has been cut during a circumcision.

As illustrated in FIGS. 2A to 2C, the present invention is related to a foreskin patch used in foreskin circumcision surgery. The foreskin patch 1 is in the form of an elongate strip, having an adhesive surface 11 on one side, further comprising a ventilation structure 12. The ventilation structure may comprise breathable meshes in one embodiment, as illustrated in FIG. 2A, and may alternatively comprise breather holes in a second embodiment, as illustrated in FIG. 2B.

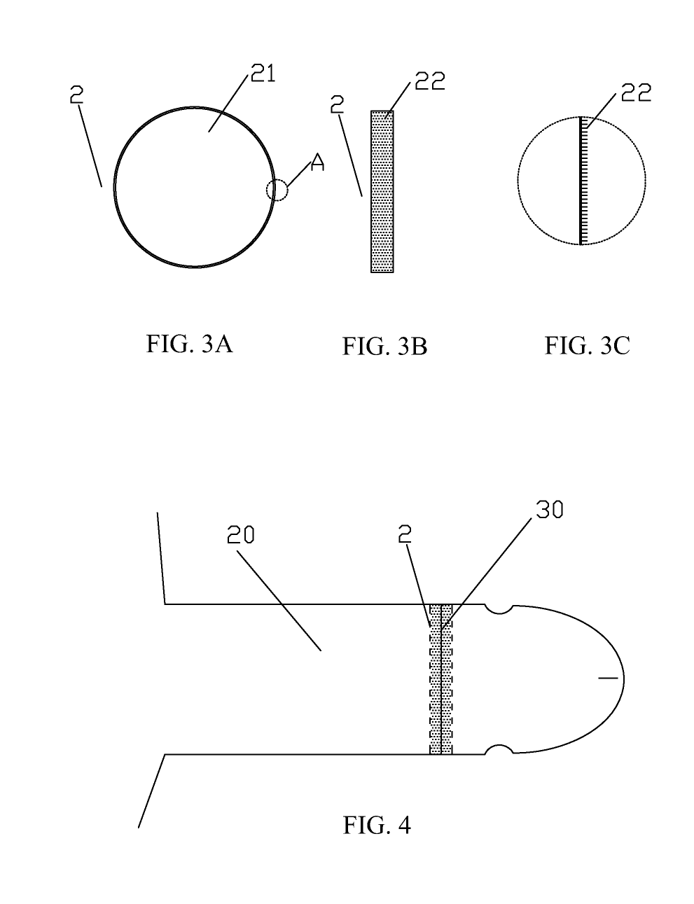

Next, referring to FIGS. 3A and 3B, a fixator 2 is shown, used in conjunction with the foreskin patch 1 for circumcision surgery. The fixator 2 is an annular sleeve, which has an accommodation space 21 to accommodate penis. A plurality of fixing pins 22 are provided on the outer surface of the fixator 2. In the embodiments illustrated in FIG. 3B and 3C, the fixing pins are needle-like structures vertically located on the outer surface of the fixator 2, and the circumferential foreskin edges resulting from removal of a section of foreskin can be fixed in close relation to each other by means of the small pins.

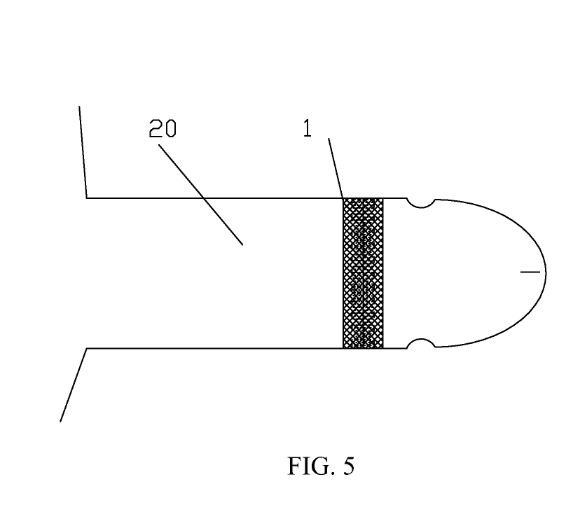

FIG. 4 illustrates a perspective view of fixator 2 in place, used to secure two circumferential foreskin edges together after removal of a middle section of foreskin. During the surgery, a strip of foreskin in the middle is cut by using one of several standard surgical techniques. The fixator 2 is sleeved on the penis 20, so that the penis is contained within the containing space 21 and the edges of the foreskin at each end is fixed by the fixing pins 22 provided on the outer surface of the fixator 2. The small pins acts to pierce the foreskin such that the two edges of each piece of foreskin are fixed with respect to each other. After the foreskin is fixed by fixator 2, the connection portions 30 of two segments of the cut foreskin are protected by using the foreskin patch 1, and the goal of enhanced fixation is achieved, as shown in FIG. 5.

Using the above-mentioned foreskin patch 1 and fixator 2, circumcision surgery is made more convenient and efficient, with faster healing times, and no stitches to remove. Additionally, surgical process is simplified. After surgery, the foreskin patch 1 can be simply torn off. Comparatively, the fixator 2 is not easily torn off, however it is made of a biological material that can be absorbed by the body. Therefore, there is no need to remove the fixator 2 after surgery, as the biological material is absorbed during the healing process. Such medical absorbability materials have been widely used.

To sum up, the present invention is expounded from the aspects of its application target, effectivity, evolutionarity and novelty, etc. The above-mentioned implementation, explained with respect to the figures, is considered to be one preferred implementation example of the invention, but should not be considered limiting. Therefore, all the structures, devices, features, etc. that are the same or similar to those of the present invention is intended to fall within the scope of the creation objective of the invention and this patent application.

What is claimed is:

1. A foreskin patch used for circumcision surgery, characterized in that the foreskin plaster is in the form of an elongate strip, having an adhesive surface on one side and further comprising a ventilation structure.

2. The foreskin patch for circumcision surgery as claimed in claim 1, characterized in that the ventilation structure comprises a plurality of breather holes or breathable meshes.

3. A fixator for use in circumcision surgery, comprising:
a fixator comprising an annular sleeve having an accommodation space to accommodate a penis, and a plurality of fixing pins located on an outer surface of the fixator.

4. The fixator of claim 3, wherein the fixing pins are needle-like structures vertically located on the outer surface of the fixator.

5. The fixator of claim 3, wherein the fixator is integrally formed from materials absorbable by a human body.

6. An apparatus for use in circumcision surgery, comprising:
a foreskin patch in the form of an elongated strip, having an adhesive surface on one side and further comprising a ventilation structure; and
a fixator comprising an annular sleeve comprising an accommodation space to accommodate a penis, and a plurality of fixing pins located on an outer surface of the fixator.

7. The apparatus of claim 6, wherein the ventilation structure comprises a plurality of breather holes.

8. The apparatus of claim 6, wherein the ventilation structure comprises a breathable mesh.

9. The apparatus of claim 6, wherein the fixing pins are needle-like structures vertically located on the outer surface of the fixator.

10. The apparatus of claim 6, wherein the fixator is integrally formed from materials absorbable by a human body.
